(19) United States
(12) Patent Application Publication
(10) Pub. No.: US 2025/0120780 A1
(43) Pub. Date: Apr. 17, 2025

(54) MECHANICAL ADVANTAGE DEVICES FOR MEDICAL DEVICES

(71) Applicant: INTUITIVE SURGICAL OPERATIONS, INC., Sunnyvale, CA (US)

(72) Inventor: Matthew Rohr Daniel, Sunnyvale, CA (US)

(21) Appl. No.: 18/916,056

(22) Filed: Oct. 15, 2024

Related U.S. Application Data
(60) Provisional application No. 63/590,695, filed on Oct. 16, 2023.

(51) Int. Cl. A61B 34/00; A61B 1/005; A61B 34/30; A61M 25/01

(52) U.S. Cl. CPC A61B 34/71; A61B 1/0057; A61B 17/29; A61B 2034/301; A61B 2034/715; A61M 25/0147

(57) ABSTRACT

A medical device includes a force transmission member, an elongate member housing first tension elements, mechanical advantage devices, and second tension elements. The first tension elements are configured to receive and transmit a tensile force. The mechanical advantage devices are configured to receive and multiply the tensile force. The second tension elements are configured to transmit the tensile force after multiplication to a distal end of the medical device. The first tension elements may include a different material than the second tension elements. The mechanical advantage devices may be pulleys or levers. A tension element may have a first end and a second distal to a mechanical advantage device and a proximal portion that is coupled to the mechanical advantage device.

### CROSS-REFERENCE TO RELATED APPLICATIONS

This application claims the priority benefit of U.S. Provisional Application No. 63/590,695, filed Oct. 16, 2023, the entire contents of which are incorporated by reference herein in its entirety.

### FIELD

The present disclosure is directed to mechanical advantage devices for medical devices.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical instruments to reach a target tissue location. Minimally invasive medical instruments include instruments such as therapeutic, diagnostic, biopsy, and surgical instruments. Medical instruments may be inserted into anatomic passageways and navigated toward a region of interest within a patient anatomy.

For flexible articulating devices driven by robotically assisted manipulators, such as instruments and/or endoscopes, articulation of the instruments and/or the endoscopes may occur via tension elements (such as drive cables) that are wound around rotating capstans. The capstans may be operatively coupled to an external device such as a robotic manipulator and receive forces/torques from the manipulator to drive the instruments and/or the endoscope. However, for applications with long path lengths within the body with tortuous anatomy, such as endoluminal applications exceeding 1 meter or longer, with the long lengths of the instruments/endoscope and associated drive cables, capstan friction from moving tensioned cables through a tortuous path can impede actuation of the surgical instrument because capstan friction increases exponentially based on bend angle. Even small increases in bend angle can multiply the force loss to such a degree that a surgical instrument functions poorly or stops working entirely, and the cumulative bend angle to reach the cecum, for example, can exceed 360 degrees. In short, the design of surgical instruments is impeded by the actuation challenges caused by capstan friction.

Instruments that can operate despite significant capstan friction are needed to enable medical procedures.

### SUMMARY

Consistent with some examples, a medical device may include a force transmission member, an elongate member housing a plurality of first tension elements, a plurality of mechanical advantage devices, and a plurality of second tension elements. The force transmission member may include one or more inputs. The force transmission member may be configured to receive forces or torques from an external device. The elongate member may be coupled to the force transmission member and may extend distally from the force transmission member. The plurality of first tension elements may be respectively coupled to the one or more inputs of the force transmission member and configured to receive and transmit a tensile force. The plurality of first tension elements may include a first material. The plurality of mechanical advantage devices may be configured to receive and multiply the tensile force. Each mechanical advantage device may be coupled to one of the plurality of first tension elements and rotatable around a respective axis of rotation. The plurality of second tension elements may be configured to transmit the tensile force after multiplication of the tensile force to a distal end of the medical device, Each second tension element may be coupled to one of the plurality of mechanical advantage devices and operatively coupled to a distal end of the medical device. The plurality of second tension elements may include a second material that is different than the first material.

In some examples, the elongate member may be flexible.

In some examples, the elongate member may be rigid.

In some examples, the first material may include one of a polymer and a metal, and the second material may include the other of the polymer and the metal.

In some examples, the metal may be at least one of tungsten and stainless steel.

In some examples, each first tension element may be coupled to the one of the plurality of mechanical advantage devices a distance from the respective axis of rotation.

In some examples, the mechanical advantage ratio of the output tensile force provided by the mechanical advantage devices may be 2:1.

In some examples, at least one of the plurality of mechanical advantage devices may be arranged in a series with a secondary mechanical advantage device and may be coupled to the secondary mechanical advantage device by an intermediate drive element to transmit and iteratively multiply the tensile force.

In some examples, the series may have a mechanical advantage ratio of at least 4:1.

In some examples, the distal end may be actuated manually when the medical device is decoupled from the external device.

In some examples, a guide housing may be provided around the plurality of mechanical advantage devices and coupled to the elongate member and to the distal end of the medical device.

In some examples, the guide housing may include guide channels to direct movement of the plurality of mechanical advantage devices.

In some examples, the plurality of mechanical advantage devices may be configured to move axially within the guide channels.

In some examples, the plurality of mechanical advantage devices may translate parallel to a longitudinal axis of the medical device within the guide channels.

In some examples, each mechanical advantage device may include a pivot at each respective axis of rotation and rotate around the pivot.

In some examples, the guide housing may include a central open passage.

In some examples, the medical device may be a surgical instrument.

In some examples, the distal end may be an end effector actuated by the plurality of second tension elements.

In some examples, the medical device may be an endoscope.

In some examples, the distal end may be an articulated bending section of the endoscope.

In some examples, the elongate member may have a length exceeding one meter.

In some examples, the plurality of mechanical advantage devices may be arranged in antagonistic pairs.

In some examples, the medical device may include four antagonistic pairs to provide bidirectional motion along four axes.

Consistent with some examples, a medical device may include a force transmission member, an elongate member housing a plurality of first tension members, a plurality of pulleys, and a plurality of second tension elements. The force transmission member may include one or more inputs, and the force transmission member may be configured to receive forces or torques from an external device. The elongate member may be coupled to the force transmission member and may extend distally from the force transmission member. The plurality of first tension elements may be respectively coupled to the one or more inputs of the force transmission member and may be configured to receive and transmit a tensile force. The plurality of pulleys may be configured to receive and multiply the tensile force. Each pulley may be rotatable around a respective axis of rotation and coupled to one of the plurality of first tension elements. The plurality of second linear tension elements may be configured to transmit the tensile force after multiplication of the tensile force to a distal end of the medical device. Each tension element may be coupled to one of the plurality of pulleys.

In some examples, the one of the plurality of first tension elements may have a first end operatively coupled to the one or more inputs of the force transmission member, a length wrapped around the pulley, and a stationary second end.

In some examples, each second tension element may be coupled to the one of the plurality of pulleys at the respective axis of rotation by a tube having a clevis interface, the tube operatively coupled to a distal end of the medical device.

In some examples, the plurality of pulleys may be configured to move axially.

In some examples, the plurality of pulleys may be configured to translate parallel to a longitudinal axis of the medical device.

Consistent with some examples, a medical device may include a force transmission member, an elongate member housing a plurality of first tension elements, a plurality of levers, and a plurality of second tension elements. The force transmission member may include one or more inputs, and the force transmission member may be configured to receive forces or torques from an external device. The elongate member may be coupled to the force transmission member and may extend distally from the force transmission member. The plurality of first tension elements may be respectively coupled to the one or more inputs of the force transmission member and may be configured to receive and transmit a tensile force. The plurality of levers may be configured to receive and multiply the tensile force. Each lever may be rotatable around a respective axis of rotation and may be coupled to one of the plurality of first tension elements at a first distance from the respective axis of rotation. A plurality of second tension elements may be configured to transmit the tensile force after multiplication of the tensile force to a distal end of the medical device. Each second tension element may be coupled to one of the plurality of levers at a second distance from the respective axis of rotation that is less than the first distance and a second end operatively coupled to a distal end of the medical device.

Consistent with some examples, a medical device may include a force transmission member, an elongate member, an end effector, a mechanical advantage device, and a tension element. The force transmission member may include one or more inputs, and the force transmission member may be configured to receive forces or torques from an external device. The elongate member may be coupled to the force transmission member and may extend distally from the force transmission member. The end effector may be operatively coupled to the force transmission member. The mechanical advantage device may be positioned between the end effector and the force transmission member. A tension element may be coupled to the end effector to drive articulation of the end effector. The tension element may have a first end that is distal to the mechanical advantage device, a second end that is distal to the mechanical advantage device, and a proximal portion that is coupled to the mechanical advantage device.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a diagram of a first robotically-assisted manipulator system, in accordance with the present disclosure.

FIG. 2A illustrates an instrument system utilizing aspects of the present disclosure.

FIG. 2B illustrates a distal portion of the instrument system of FIG. 2A with an extended example of an instrument in accordance with the present disclosure.

FIG. 3 is a perspective view of a manipulator system in accordance with the present disclosure.

FIG. 4 is a schematic view of a manipulator system in accordance with the present disclosure.

FIG. 5 is a perspective view of a medical device according to an embodiment.

FIG. 6 is a perspective view of a distal end portion of the medical device of FIG. 5.

FIG. 7 is a perspective view of a medical device including mechanical advantage devices, where the mechanical advantage devices are pulleys, in accordance with the present disclosure.

FIG. 8 is a partial cross-sectional view of the medical device of FIG. 7.

FIG. 9 is a perspective view of a proximal bulkhead of the medical device of FIGS. 7 and 8.

FIG. 10 is a perspective view of a housing of the mechanical advantage device of FIGS. 7 and 8.

FIG. 11 is a perspective view of a distal bulkhead of the medical device of FIGS. 7 and 8.

FIG. 12 is a perspective view of a tube having a clevis interface of the medical device of FIGS. 7 and 8.

FIG. 13 is a cross-sectional view of a medical device including a plurality of mechanical advantage devices arranged in a compound series, in accordance with the present disclosure.

FIG. 14 is a perspective view of a medical device including a plurality of mechanical advantage devices arranged in a linear series, in accordance with the present disclosure.

FIG. 15 is a cross-sectional view of a medical device including mechanical advantage devices, where the mechanical advantage devices are levers, in in accordance with the present disclosure.

FIG. 16 is a schematic illustration of a method of assembling a medical device including a plurality of mechanical advantage devices, in accordance with the present disclosure.

FIG. 17 is a schematic illustration of a method of actuating a medical device including a plurality of mechanical advantage devices, in accordance with the present disclosure.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

In the following detailed description of the aspects of the invention, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the invention. And, to avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

Mechanical advantage devices are provided that allow a mechanical force to be multiplied before being delivered to a distal end of a surgical instrument (where, for example, an end effector and/or optional wrist mechanism may be attached). The multiplied mechanical force is sufficient to allow effective actuation of the surgical instrument despite, for example, capstan friction. Exemplary mechanical advantage devices, such as pulleys and levers, do not merely incidentally provide a mechanical advantage while, for example, redirecting a force in a different direction. Instead, the mechanical advantage devices have the sole purpose of multiplying a mechanical force before that force is utilized within, for example, an end effector or wrist mechanism. The mechanical advantage may have a ratio of 2:1. In some arrangements, a plurality of mechanical advantage devices may be arranged in a series to increase the mechanical advantage further.

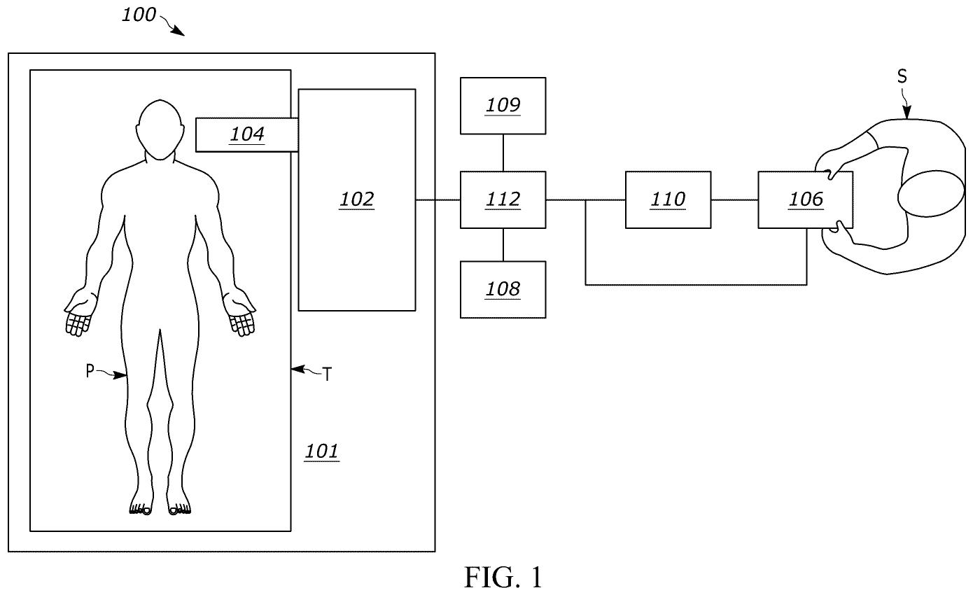

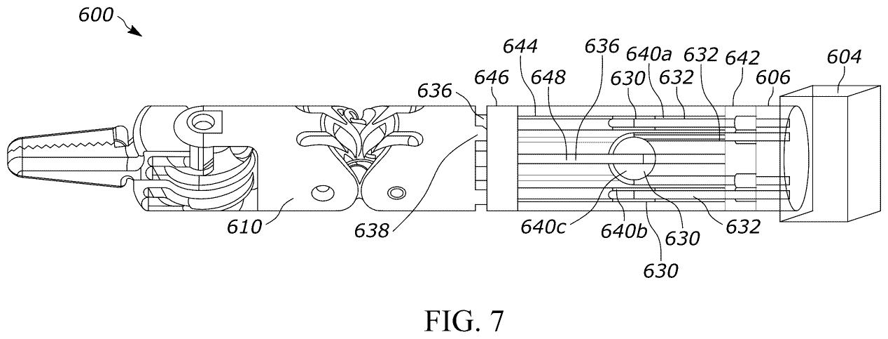

Turning to the drawings, FIGS. 1-6 of the drawings illustrate systems and medical instruments that can be adapted for use with mechanical advantage devices, and FIGS. 7-14 illustrate implementations and features of mechanical advantage devices.

FIG. 1 illustrates an embodiment of a robotically-assisted manipulator system for use with the rotary to linear force articulation members described herein. The manipulator system may be used, for example, in surgical, diagnostic, therapeutic, biopsy, or non-medical procedures, and is generally indicated by the reference numeral 100. As shown in FIG. 1, a robotically-assisted manipulator system 100 may include one or more manipulator assemblies 102 for operating one or more medical instrument systems 104 in performing various procedures on a patient P positioned on a table T in a medical environment 101. For example, the manipulator assembly 102 may drive catheter or end effector motion, may apply treatment to target tissue, and/or may manipulate control members. The manipulator assembly 102 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. An operator input system 106, which may be inside or outside of the medical environment 101, generally includes one or more control devices for controlling manipulator assembly 102. Manipulator assembly 102 supports medical instrument system 104 and may optionally include a plurality of actuators or motors that drive inputs on medical instrument system 104 in response to commands from a control system 112. The actuators may optionally include drive systems that when coupled to medical instrument system 104 may advance medical instrument system 104 into a naturally or surgically created anatomic orifice. Other drive systems may move the distal end of medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). The manipulator assembly 102 may support various other systems for irrigation, treatment, or other purposes. Such systems may include fluid systems (including, for example, reservoirs, heating/cooling elements, pumps, and valves), generators, lasers, interrogators, and ablation components.

Robotically-assisted manipulator system 100 also includes a display system 110 for displaying an image or representation of the surgical site and medical instrument system 104 generated by an imaging system 109 which may include an endoscopic imaging system. Display system 110 and operator input system 106 may be oriented so an operator O can control medical instrument system 104 and operator input system 106 with the perception of telepresence. A graphical user interface may be displayable on the display system 110 and/or a display system of an independent planning workstation.

In some examples, the endoscopic imaging system components of the imaging system 109 may be integrally or removably coupled to medical instrument system 104. However, in some examples, a separate endoscope, attached to a separate manipulator assembly may be used with medical instrument system 104 to image the surgical site. The endoscopic imaging system 109 may be implemented as hardware, firmware, software, or a combination thereof which interact with or are otherwise executed by one or more computer processors, which may include the processors of the control system 112.

Robotically-assisted manipulator system 100 may also include a sensor system 108. The sensor system 108 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 104. The sensor system 108 may also include temperature, pressure, force, or contact sensors or the like.

Robotically-assisted manipulator system 100 may also include a control system 112. Control system 112 includes at least one memory 116 and at least one computer processor 114 for effecting control between medical instrument system 104, operator input system 106, sensor system 108, and display system 110. Control system 112 also includes programmed instructions (e.g., a non-transitory machine-readable medium storing the instructions) to implement a procedure using the robotically-assisted manipulator system including for navigation, steering, imaging, engagement feature deployment or retraction, applying treatment to target tissue (e.g., via the application of energy), or the like.

Control system 112 may optionally further include a virtual visualization system to provide navigation assistance to operator O when controlling medical instrument system 104 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 112 may use a pre-operative image to locate the target tissue (using vision imaging techniques and/or by receiving user input) and create a pre-operative plan, including an optimal first location for performing treatment. The pre-operative plan may include, for example, a planned size to expand an expandable device, a treatment duration, a treatment temperature, and/or multiple deployment locations.

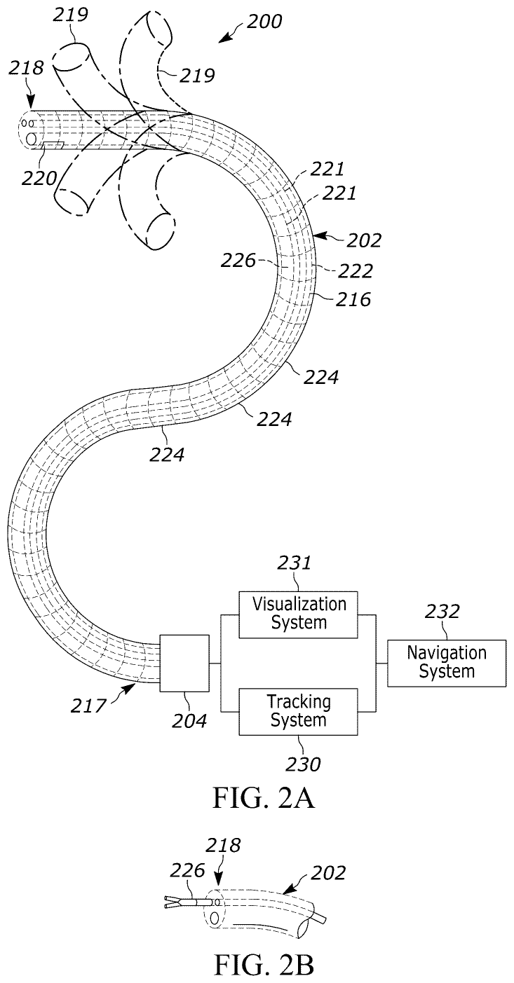

FIG. 2A is a simplified diagram of a medical instrument system 200 according to some embodiments. In some embodiments, medical instrument system 200 may be used in an image-guided medical procedure. In some examples, medical instrument system 200 may be used for non-teleoperational exploratory procedures or in procedures involving traditional manually operated medical instruments, such as endoscopy.

Medical instrument system 200 includes elongate flexible device 202, such as a flexible catheter or endoscope, coupled to a drive unit 204. Elongate device 202 includes a flexible body 216 having proximal end 217 and distal end, or tip portion, 218. In some embodiments, flexible body 216 has an approximately 14-20 mm outer diameter. Other flexible body outer diameters may be larger or smaller.

Medical instrument system 200 optionally includes a tracking system 230 for determining the position, orientation, speed, velocity, pose, and/or shape of distal end 218 and/or of one or more segments 224 along flexible body 216 using one or more sensors and/or imaging devices. The entire length of flexible body 216, between distal end 218 and proximal end 217, may be effectively divided into segments 224. Tracking system 230 may optionally be implemented as hardware, firmware, software or a combination thereof which interact with or are otherwise executed by one or more computer processors, which may include the processors of control system 112 in FIG. 1.

Tracking system 230 may optionally track distal end 218 and/or one or more of the segments 224 using a shape sensor 222. In some embodiments, tracking system 230 may optionally and/or additionally track distal end 218 using a position sensor system 220, such as an electromagnetic (EM) sensor system. In some examples, position sensor system 220 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

Flexible body 216 includes one or more channels 221 sized and shaped to receive one or more medical instruments 226. In some embodiments, flexible body 216 includes two channels 221 for separate instruments 226, however, a different number of channels 221 may be provided. FIG. 2B is a simplified diagram of flexible body 216 with medical instrument 226 extended according to some embodiments. In some embodiments, medical instrument 226 may be used for procedures such as surgery, biopsy, ablation, illumination, irrigation, or suction. Medical instrument 226 can be deployed through channel 221 of flexible body 216 and used at a target location within the anatomy. Medical instrument 226 may include, for example, image capture devices, biopsy instruments, ablation instruments, catheters, laser ablation fibers, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electrosurgical electrodes, transducers, sensors, and/or the like. Medical instrument 226 may be advanced from the opening of channel 221 to perform the procedure and then retracted back into the channel when the procedure is complete. Medical instrument 226 may be removed from proximal end 217 of flexible body 216 or from another optional instrument port (not shown) along flexible body 216. The medical instrument 226 may be used with an image capture device (e.g., an endoscopic camera) also within the flexible body 216. Alternatively, the medical instrument 226 may itself be the image capture device.

Medical instrument 226 may additionally house cables, linkages, or other actuation controls (not shown) that extend between its proximal and distal ends to controllably the bend distal end of medical instrument 226. Flexible body 216 may also house cables, linkages, or other steering controls (not shown) that extend between drive unit 204 and distal end 218 to controllably bend distal end 218 as shown, for example, by broken dashed line depictions 219 of distal end 218. In some examples, at least four cables are used to provide independent “up-down” steering to control a pitch motion of distal end 218 and “left-right” steering to control a yaw motion of distal end 218. In embodiments in which medical instrument system 200 is actuated by a robotically-assisted assembly, drive unit 204 may include drive inputs that removably couple to and receive power from drive elements, such as actuators, of the teleoperational assembly. In some embodiments, medical instrument system 200 may include gripping features, manual actuators, or other components for manually controlling the motion of medical instrument system 200. The information from tracking system 230 may be sent to a navigation system 232 where it is combined with information from visualization system 231 and/or the preoperatively obtained models to provide the physician or other operator with real-time position information.

Other configurations of teleoperated manipulator systems are also contemplated, such as systems configured for multi-port or single-port procedures. For example, the embodiments described herein may be used with a da Vinci® Surgical System, such as the da Vinci X®, Xi®, or SP® Surgical Systems, all commercialized by Intuitive Surgical, Inc., of Sunnyvale, California.

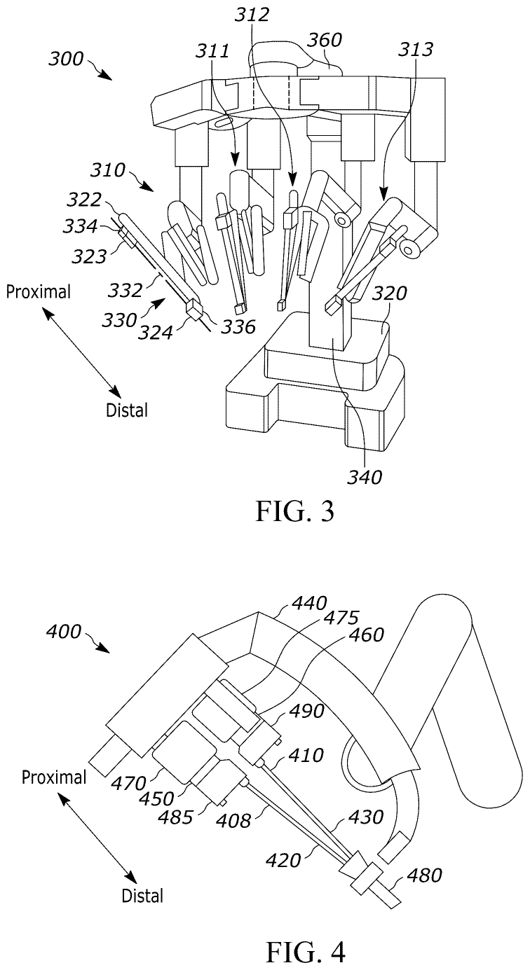

FIG. 3 illustrates an example embodiment of a manipulator system 300 that may be used as part of the manipulator system 100. The manipulator system 300 includes a base 320, a main column 340, and a main boom 360 connected to main column 340. Manipulator system 300 also includes a plurality of manipulator arms 310, 311, 312, 313, which are each connected to main boom 360. The manipulator arms 310, 311, 312, and 313, may be used as the manipulator assemblies 102. Manipulator arms 310, 311, 312, 313 each include an instrument mount portion 322 to which an instrument 330 may be mounted, which is illustrated as being attached to manipulator arm 310. While the manipulator system 300 depicts four manipulator arms, various embodiments may include more or fewer manipulator arms.

Instrument mount portion 322 may include a drive assembly 323 and a cannula mount 324, with a transmission mechanism 334 of the instrument 330 connecting with the drive assembly 323, according to an embodiment. Cannula mount 324 is configured to hold a cannula 336 through which a shaft 332 of instrument 330 may extend to a surgery site during a surgical procedure. Drive assembly 323 contains a variety of drive and other mechanisms that are controlled to respond to input commands at the operator input system 106 and transmit forces to the transmission mechanism 334 to actuate the instrument 330. Although the embodiment of FIG. 3 shows an instrument 330 attached to only manipulator arm 310 for ease of viewing, an instrument may be attached to any and each of manipulator arms 310, 311, 312, 313.

FIG. 4 illustrates an example embodiment of a manipulator system 400 that may be used as part of the manipulator system 100. In FIG. 4, a portion of a manipulator arm 440 of the manipulator system 400 is shown with two instruments 408, 410 in an installed position. The schematic illustration of FIG. 4 depicts only two instruments for simplicity, but more than two instruments may be mounted in an installed position at the manipulator system 400 as those having ordinary skill in the art are familiar. Each instrument 408, 410 includes a shaft 420, 430 having at a distal end a moveable end effector or an endoscope, camera, or other sensing device, and may or may not include a wrist mechanism (not shown) to control the movement of the distal end.

In the embodiment of FIG. 4, the distal end portions of the instruments 408, 410 are received through a single port structure 480 to be introduced into the patient. As shown, the port structure includes a cannula and an instrument entry guide inserted into the cannula. Individual instruments are inserted into the entry guide to reach a surgical site.

Transmission mechanisms 485, 490 are disposed at a proximal end portion of each shaft 420, 430 and connect through a sterile adaptor 450, 460 with drive assemblies 470, 475, which contain a variety of internal mechanisms (not shown) that are controlled by a controller (e.g., at a control cart of a surgical system) to respond to input commands at a surgeon side console of a surgical system to transmit forces to the force transmission mechanisms 485, 490 to actuate instruments 408, 410.

The manipulator systems described herein are not limited to the embodiments of FIGS. 1, 2, 3, and 4, and various other teleoperated, computer-assisted manipulator configurations may be used with the embodiments described herein. The diameter or diameters of an instrument shaft and end effector are generally selected according to the size of the cannula with which the instrument will be used and depending on the surgical procedures being performed.

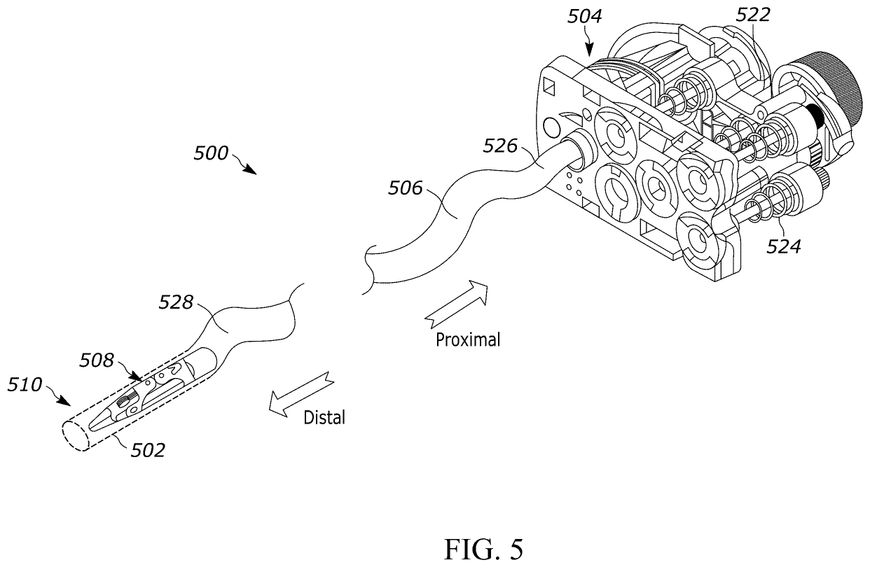

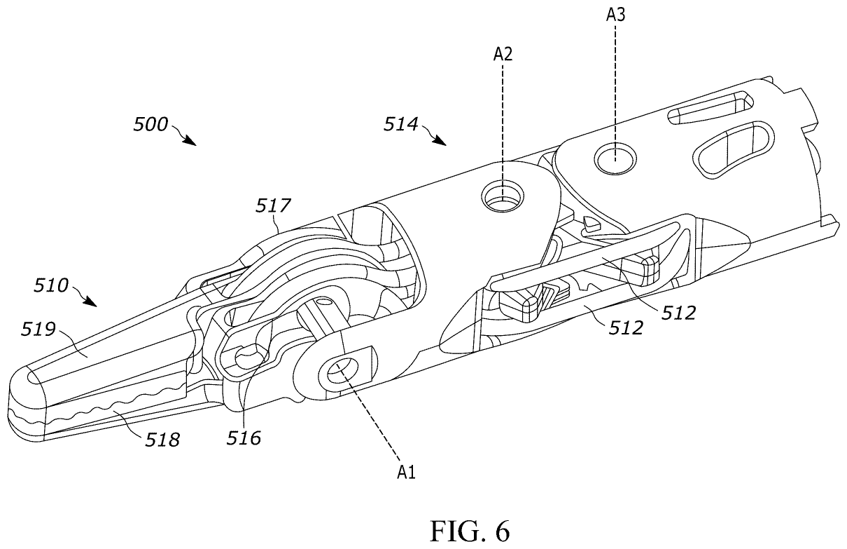

FIGS. 5 and 6 are various views of a medical device 500, according to an embodiment. In some embodiments, the medical device 500 or any of the components therein are optionally parts of an instrument for a surgical system that performs surgical procedures, and which surgical system can include a manipulator unit, a series of kinematic linkages, a series of cannulas, or the like. The medical device 500 (and any of the instruments described herein) can be used in any suitable surgical system, such as the manipulator system 100 or the manipulator system 200 shown and described above. The medical device 500 may be used as the medical instruments 226, 330, 408, and 410 described above. As shown in FIG. 5, the medical device 500 defines (or is included within) a distal boundary (or footprint) 502 that corresponds to a cannula size, or a size to fit within a working channel of an elongate flexible device (such as a flexible catheter or endoscope), or other size dictated by the surgical environment. An elongate flexible device is useful for anatomic passageways, such as the gastrointestinal (GI) tract, that are long and circuitous. Surgical instruments used for GI procedures such as Endoscopic Submucosal Dissection (ESD) and Endoscopic Mucosal Resection (EMR) may function through long, flexible working channels of standard or customized endoscopes or otherwise along such endoscopes, which can reach lengths exceeding 1.6 meters. The distal boundary 502 can be a cylindrical shape having any suitable nominal diameter (e.g., 8 mm, 5 mm or any size therebetween). The medical device 500 includes a force transmission mechanism 504, a shaft 506, an optional distal wrist assembly 508, a distal end effector 510, and a set of tension elements 512 (which can be, for example, a cable, band, or the like).

The medical device 500 can include multiple tension elements 512. For example, in some embodiments, the medical device 500 can include two tension elements 512 with each tension element 512 having two segments extending along the shaft 506 of the instrument, thereby forming four proximal end portions. Referring to FIG. 6, respective tension elements 512 may be routed through a wrist assembly 514 and wrapped about respective pulleys 516 or 517 of the tool members 518, 519. Each tension element 512 has two tension element segments along the shaft 506 with two proximal end portions that, when moved in opposite directions, can (among other things) cause rotation of the respective tool member 518 or 519 about the axis A1. This arrangement can be referred to as a “four cable” wrist and changing the pitch, yaw, or grip of the medical device 500 can be performed by manipulating the four proximal end portions of the tension elements 512 in a manner similar to that shown and described in U.S. Patent Publication No. 2020/0390430 incorporated herein by reference above. In other embodiments, the medical device 500 can include four separate tension elements 512 with two separate tension elements coupled to the pulley 516 of the tool member 518 and two separate tension elements coupled to the pulley 517 of the tool member 518, thereby creating four proximal tension element end portions. In some embodiments, the medical device 500 can include more than two or four tension elements 512 and more than four proximal tension element end portions. The tension elements 512 can be, for example, cables, bands, or the like that couple the force transmission mechanism 504 to the distal wrist assembly 508 and end effector 510. In some embodiments, the tension elements 512 can be constructed from a polymer.

The medical device 500 is configured such that movement of one or more of the tension elements 512 produces rotation of the end effector 510 about a first rotation axis A1 (see FIG. 6, which functions as a yaw axis, the term yaw is arbitrary), rotation of the wrist assembly 508 about a second rotation axis A2 and/or optionally about a third rotation axis A3 (which functions as a pitch axis, the term pitch is arbitrary), a cutting rotation of the tool members of the end effector 510 about the first rotation axis A1, or any combination of these movements. Changing the pitch or yaw of the medical device 500 can be performed by manipulating the tension elements 512 in a similar manner as that described with reference to the device 2400 described in copending International Patent Application Serial No. PCT/US2022/039942, entitled “Surgical Instrument Cable Control and Routing Structures,” the disclosure of which is incorporated herein by reference in its entirety.

As shown in FIG. 5, the proximal force transmission mechanism 504 includes a set of drive components such as capstans 522 and 524 that rotate or “wind” a proximal portion of any of the tension elements 512 to produce the desired tension element movement. In some embodiments, two proximal ends of a tension element 512, which are associated with opposing directions of a single degree of freedom, are connected to two independent drive capstans 522 and 524. This arrangement, which is generally referred to as an antagonist drive system, allows for independent control of the movement of (e.g., pulling in or paying out) each of the ends of the tension elements 512. The force transmission mechanism 504 produces movement of the tension elements 512, which operates to produce the desired articulation movements (pitch, yaw, or grip) at the wrist assembly 508 and end effector 510. Accordingly, the force transmission mechanism 504 includes components to move a first proximal end portion of the tension element 512 via the first capstan 522 in a first direction (e.g., a proximal direction) and to move a second proximal end portion of the tension element 512 via the second capstan 524 in a second opposite direction (e.g., a distal direction). The force transmission mechanism 504 can also move both proximal end portions of the tension element 512 in the same direction. In this manner, the force transmission mechanism 504 can maintain the desired tension within the tension elements 512.

In some embodiments, the force transmission mechanism 504 can include any of the assemblies or components described in International Patent Application Serial No. PCT/US2022/039942, entitled “Surgical Instrument Cable Control and Routing Structures,” the disclosure of which is incorporated herein by reference in its entirety. In other embodiments, however, any of the medical devices described herein can have the two ends of a tension elements wrapped about a single capstan. This alternative arrangement, which is generally referred to as a self-antagonist drive system, operates the two ends of the tension element using a single drive motor.

Moreover, although the force transmission mechanism 504 is shown as including capstans, in other embodiments, a force transmission mechanism can include one or more linear actuators that produce translation (linear motion) of a portion of the cables. Such force transmission mechanisms can include, for example, a gimbal, a lever, or any other suitable mechanism to directly pull (or release) an end portion of any of the cables. For example, in some embodiments, the proximal force transmission mechanism 504 can include any of the proximal force transmission mechanisms or components described in U.S. Patent Application Pub. No. US 2015/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disk Wrist Joint,” each of which is incorporated herein by reference in its entirety.

The shaft 506 can be any suitable elongated shaft that is coupled to the force transmission mechanism 504 and to the optional wrist assembly 508 (when present) or the end effector 510. Specifically, the shaft 506 includes a proximal portion 526 that is coupled to the force transmission mechanism 504, and a distal portion 528 that is coupled to the optional wrist assembly 508 or to the end effector 510. The shaft 506 defines a passageway or series of passageways through which the tension elements 512 and other components (e.g., electrical wires, ground wires, or the like) can be routed from the force transmission mechanism 504 to the wrist assembly 508. In some embodiments, the shaft 506 may be a substantially rigid member, while in other embodiments, the shaft 506 may be a flexible member.

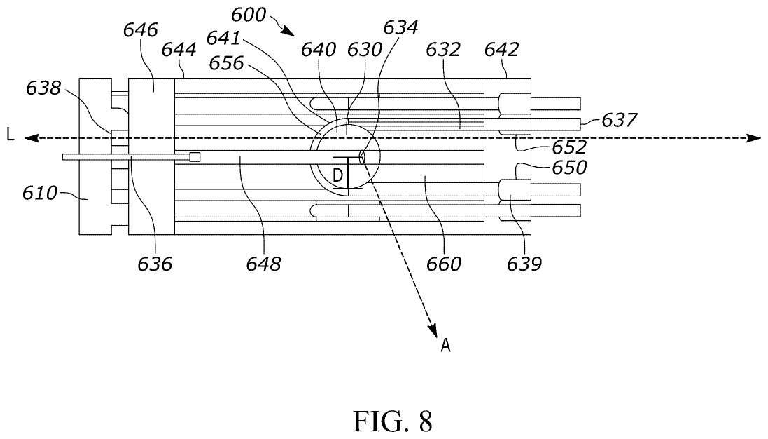

FIGS. 7 and 8 illustrate a medical device 600 including a plurality of mechanical advantage devices 630. The medical device 600 may be, for example, a surgical instrument or an endoscope. The plurality of mechanical advantage devices 630 may be configured to fit within, for example, an endoscope having an outer diameter between 16 mm and 20 mm or an instrument having an outer diameter between 4 mm and 6 mm. Features shared with the device 500 of FIGS. 5 and 6 are referred to by the same reference number increased by one hundred. After their initial introduction, common features are not described in substantial detail for subsequent arrangements. Unique features are identified by unique reference numbers.

Like the medical device 500 shown in FIGS. 5 and 6, the medical device 600 includes a force transmission member 604 including one or more inputs. The force transmission member 604 is configured to receive forces or torques from an external device. An elongate member 606 is coupled to the force transmission member 604 and extends distally from the force transmission member 604. The elongate member 606 may be flexible or may be rigid. The elongate member 606 may have a length exceeding one meter, for example if the medical device is intended for use within the gastrointestinal tract. The elongate member 606 houses a plurality of first tension elements 632 respectively coupled to the one or more inputs of the force transmission member 604 and configured to receive and transmit a tensile force. The first tension elements 632 are comparable to the tension elements 512 except that the first tension elements 632 terminate before reaching a distal end 638 of the medical device 600, as shown in FIG. 7. For example, the first tension elements 632 terminate proximally to the end effector 610 and wrist mechanisms depicted in FIG. 7.

Turning to FIGS. 7 and 8, each of the plurality of mechanical advantage devices 630 are configured to receive and multiply the tensile force when respective first tension elements 632 are actuated. Each mechanical advantage device 630 is coupled to one of the plurality of first tension elements 632 and is rotatable around a respective axis of rotation A by, for example, a pivot pin 634 (shown in FIG. 8). Specifically, in the arrangement shown in FIGS. 7 and 8, the plurality of mechanical advantage devices 630 are movable pulleys and the axis of rotation A is at the center of the movable pulleys, which is where the pivot 634 is disposed. The pulleys are rotatable about their respective axes of rotation A and the pulleys are also axially movable relative to the elongate member 606 as discussed below.

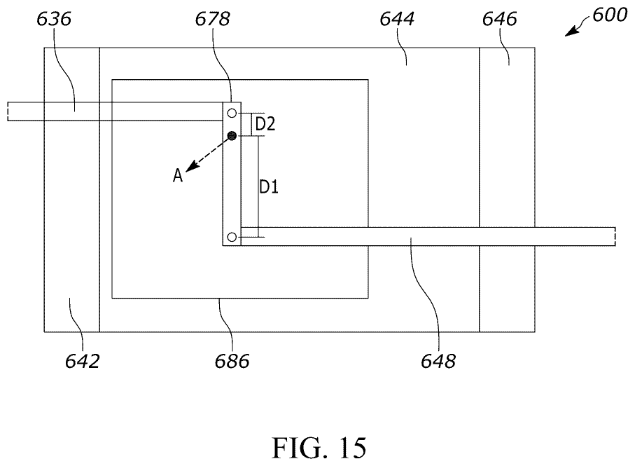

As discussed below with respect to FIG. 15, in other arrangements, the plurality of mechanical advantage devices 630 may be levers. As shown in FIGS. 7 and 8, a plurality of second tension elements 636 are configured to transmit the tensile force to a distal end 638 of the medical device (e.g., to a distal end effector 610) after multiplication of the tensile force by the mechanical advantage devices 630. As shown in FIG. 8, each first tension element 632 is coupled to one of the plurality of mechanical advantage devices 630 a distance D from the respective axis of rotation A. In the arrangement shown in FIGS. 7 and 8, each first tension element 632 is wound around a mechanical advantage device 630 that is a pulley. As best shown in FIG. 8, each first tension element 632 has a first end 637 that is proximal to the mechanical advantage device 630, a second end 639 that is proximal to the mechanical advantage device 630, and a distal portion 641 that is coupled to the mechanical advantage device 630.

Each second tension element 636 is operatively coupled to the distal end 638 of the medical device 600. As an example, the distal end 638 may include an end effector 610 and/or wrist mechanism that is actuated by the plurality of second tension elements 636 as shown in FIG. 7. As another example, the distal end may be an articulated bending section of an endoscope. When the medical device 600 is decoupled from the external device, the distal end 638 can be optionally actuated manually. This is useful in, for example, a situation where a power supply to the external device fails.

A benefit of having both a plurality of first tension elements 632 and a plurality of second tension elements 636 as opposed to a single set of tension elements 512 is that different materials may be used that optimize their properties relative to their location within the medical device 600. The plurality of first tension elements 632 may include a first material, and the plurality of second tension elements 636 may include a second material that is different than the first material. For example, the first material may include one of a polymer and a metal, and the second material may include the other of the polymer and the metal. The polymer may be, for example, at least one of an ultra-high molecular weight polyethylene or nylon. The metal may, for example, be at least one of tungsten and stainless steel. Metal is typically stiffer than a polymer but may have disadvantages for wrapping around small bends, whereas a polymer may have a lower coefficient of friction, and the ability to wrap under smaller bend radii but provides less stiffness. The design of the medical device 600 impacts which material properties are most important, and thus the design of the medical device 600 can drive the choice of materials for the plurality of first tension elements 632 and a plurality of second tension elements 636. In some arrangements, the plurality of first tension elements 632 and the plurality of second tension elements 636 may include the same material if similar material properties are optimal throughout the medical device 600.

The plurality of mechanical advantage devices 630 may be arranged in antagonistic pairs. For example, in the arrangement shown in FIG. 7, pulleys 640a and 640b which are arranged parallel to one another may be an antagonistic pair. For example, movement of one pulley of the antagonistic pair (e.g., pulley 640a) in a first direction results in an equidistant movement of the other paired pulley (e.g., pulley 640b) in an opposite direction. To achieve this, the second tension element 636 coupled to the first pulley 640a may be connected to the second tension element 636 coupled to the second pulley 640b via, for example, a cord, cable, or connection structure of the end effector 610. The antagonistic pair of pulleys 640a and 640b can thus provide bidirectional movement along an axis of, for example, an end effector 610. If bidirectional movement along a greater number of axes is desired, additional antagonistic pairs may be added. For example, the medical device 600 may include four antagonistic pairs of mechanical advantage devices 630 to provide bidirectional motion along four axes.

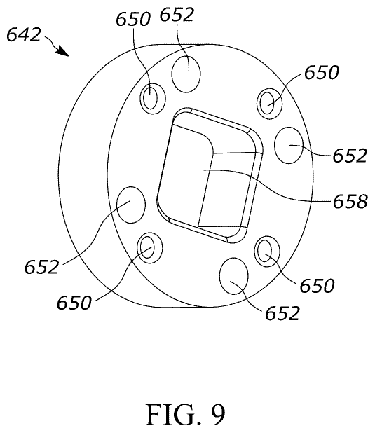

To provide the functionality described above, the medical device 600 further includes a proximal bulkhead 642 to which and through which the plurality of first tension elements 632 are connected and extend, a guide housing 644 within which the mechanical advantage devices 630 are positioned, a distal bulkhead 646 adjacent the guide housing 644, and tubes 648 extending through the distal bulkhead 646 and connected to the plurality of second tension elements 636. The proximal bulkhead 642, guide housing 644, distal bulkhead 646, and tubes 648 are depicted, respectively, in FIGS. 9-12 and described in additional detail below.

As shown in FIG. 9, the proximal bulkhead 642 includes a plurality of connection apertures 650 and a plurality of proximal passageways 652. As shown in FIG. 8, the first end 637 of the first tension element 632 is secured within or to the connection aperture 650 by, for example, an adhesive, friction fit, or other means of connection. As shown in FIG. 8, a length 656 (e.g., of portion 641) is wrapped around a mechanical advantage device 630 (here, a pulley), and then extends through a proximal passageway 652 to operatively couple to the force transmission member 604. Returning to FIG. 9, the proximal bulkhead 642 includes a proximal open center 658 through which, for example, drive cables, electrical cables, or a fluid channel can pass if used in a medical device that is an instrument. If used in a medical device that is an endoscope, the proximal open center 658 allows passage of, for example, working channels, vision system electronics, shape fiber, and fluid channels.

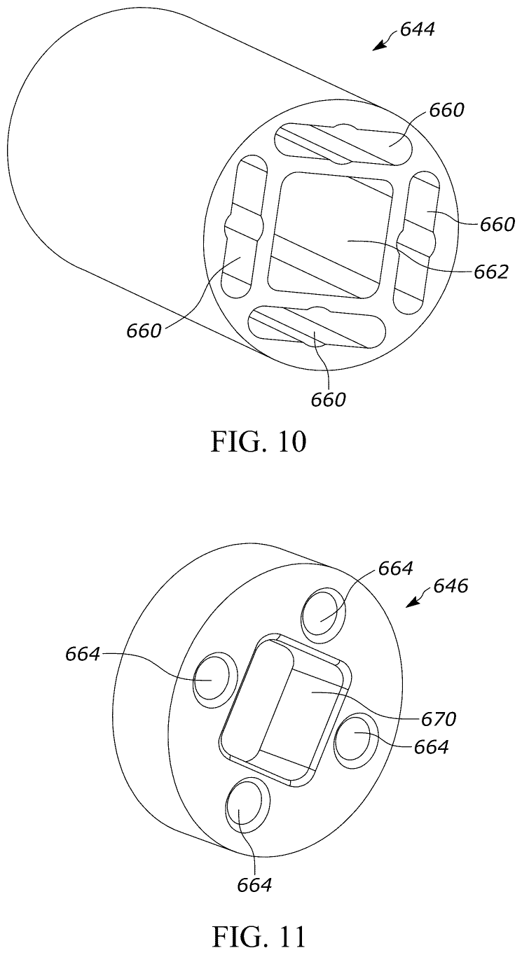

As shown in FIG. 7, the guide housing 644 is provided around the plurality of mechanical advantage devices 630 and is coupled to the elongate member 606 (similar to shaft 506 in FIG. 5) and to the distal end 638 of the medical device 600 (shown in FIG. 7). As shown in FIG. 10, the guide housing 644 includes guide channels 660 to direct movement of the plurality of mechanical advantage devices 630. Each of the plurality of mechanical advantage devices 630 is provided within a guide channel 660. The guide channels 660 align the orientations of the respective mechanical advantage devices 630 and prevent the mechanical advantage devices 630 from becoming entangled with one another. In the arrangement shown, the guide channels 660 have a cross-section that is complementary to the cross-section of a pulley with extra space (e.g., notches or grooves) provided for the pivot pin 634 of each pulley. The guide housing 644 includes a central open passage 662 through which, for example, drive cables, electrical cables, or a fluid channel can pass if used in a medical device that is an instrument. If used in a medical device that is an endoscope, the central passage 662 allows passage of, for example, working channels, vision system electronics, shape fiber, and fluid channels.

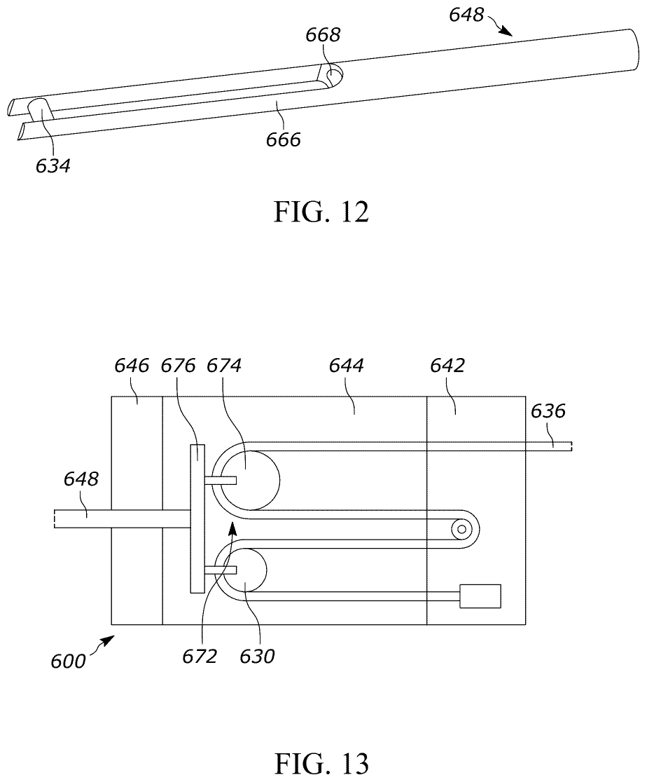

As shown in FIG. 11, the distal bulkhead 646 includes a plurality of distal passageways 664 through which the plurality of first tension elements 632 operate. One of the plurality of second tension elements 636 is coupled to each mechanical advantage device 630 by a tube 648 shown in FIG. 12. The tube 648 includes a clevis interface 666 to connect to the pivot pin 634 at a respective axis of rotation A. The clevis interface 666 allows the mechanical advantage device 630 to which it is connected to rotate around the pivot pin 634 without obstruction. Distal to the clevis interface 666, the tube 648 includes a central path 668 into which the respective second tension element 636 can be inserted and secured to the tube 648. Each tube 648 is inserted through one of the plurality of distal passageways 664 of the distal bulkhead 646 to operatively couple to the distal end 638 of the medical device 600 via a respective second tension element 636. For example, if the distal end 638 is an end effector 610, the second end 639 of the second tension element 636 may be fixed to a structure within the end effector 610 to facilitate movement of the end effector 610. As shown in FIG. 11, the distal bulkhead 646 further includes a distal open center 670 through which, for example, drive cables, electrical cables, or a fluid channel can pass if used in a medical device that is an instrument. If used in a medical device that is an endoscope, the distal open center 670 allows passage of, for example, working channels, vision system electronics, shape fiber, and fluid channels.

Returning to FIG. 8, movement of the first tension element 632 in a first direction away from the distal end 638 (as, for example, a response to the force transmission member 604) causes the mechanical advantage device 630 to be displaced axially away from the distal end 638 in the first direction. When the mechanical advantage device 630 is a pulley, because the second end 639 of the first tension element 632 is stationary, movement of the first end 637 causes the pulley to rotate around the axis A and be displaced proximally. The guide channel 660 of the guide housing 644 prevents movement of the mechanical advantage device 630 in any non-axial direction. The guide channel 660 ensures that the mechanical advantage device 630 translates parallel to a longitudinal axis of the medical device 600. The mechanical advantage device 630 thereby exerts a force on the second tension element 636 via tube 648 and consequently on, for example, the end effector 610 to actuate the medical device 600.

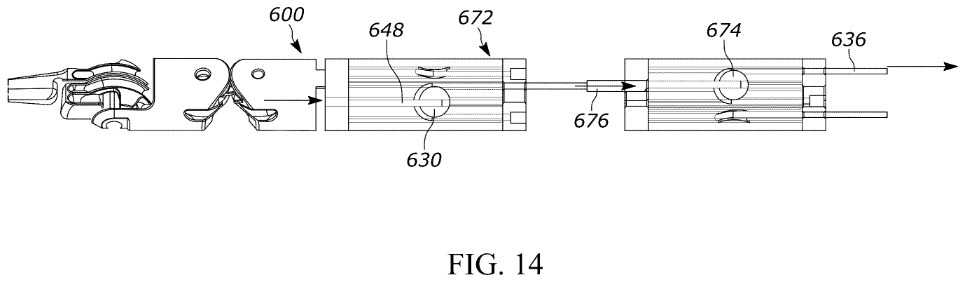

Turning to FIGS. 13 and 14, the medical device 600 may optionally have at least one of the plurality of mechanical advantage devices 630 arranged in a series 672 with a secondary mechanical advantage device 674 and may be coupled to the secondary mechanical advantage device 674 by an intermediate drive element 676 to transmit and iteratively multiply the tensile force. In the arrangement shown in FIGS. 13 and 14, the secondary mechanical advantage device 674 is a pulley. In FIG. 13, the one of the plurality of mechanical advantage devices 630 and the secondary mechanical advantage device 674 are illustrated in the same cross-sectional plane. In FIG. 14, the secondary mechanical advantage device 674 may be is positioned proximal to the mechanical advantage device 630 in order to minimize an outer diameter of the medical device 600. The series 672 may have a mechanical advantage ratio of at least 4:1. In addition, where both the mechanical advantage device 630 and the secondary mechanical advantage device 674 include pulleys, the pulleys may have the same size as each other or may have different sizes from each other.

FIG. 15 illustrates a medical device 600 using levers 678 as the plurality of mechanical advantage devices 630. For clarity, FIG. 15 illustrates only a single exemplary lever 678 although multiple levers would be used in the device 600. Each lever 678 is rotatable around the respective axis of rotation A and is coupled to one of the plurality of first tension elements 632 at a first distance D1 from the respective axis of rotation A. Each lever 678 is further coupled to one of the plurality of second tension elements 636 a second distance D2 from the respective axis of rotation A. The second distance D2 is less than the first distance D1 in order for the lever 678 to provide a mechanical advantage. With this arrangement, the proximal bulkhead 642 does not need to have connection apertures 650. Instead, the pivot pin 634 may be securable to the guide housing 644. Further, rather than including guide channels 660, the guide housing 644 may include a recess 686 for each lever 678 to rotate within.

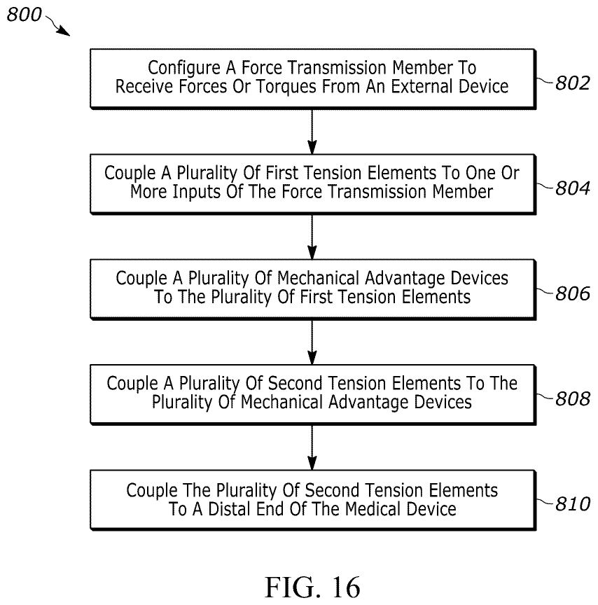

FIG. 16 illustrates a method 800 of assembly of a medical device 600. At box 802, the method 800 includes configuring a force transmission member 604 to receive forces or torques from an external device. At box 804, the method 800 includes coupling a plurality of first tension elements 632 to one or more inputs of the force transmission member 604. At box 806, the method 800 includes coupling a plurality of mechanical advantage devices 630 to the plurality of first tension elements 632. At box 808, the method 800 includes coupling a plurality of second tension elements 636 to the plurality of mechanical advantage devices 630. At box 810, the method includes coupling the plurality of second tension elements 636 to a distal end 638 of the medical device 600. The method 800 may further include housing the plurality of first tension elements 632 within a flexible elongate member 606. Alternately, the method 800 may further include housing the plurality of first tension elements 632 within a rigid elongate member 606. The method 800 may include selecting a plurality of first tension elements 632 that comprise a first material and selecting a plurality of second tension elements 636 that comprise a second material that is different than the first material. Further, assembly of any of the various features and structures described above may be included in method 800.

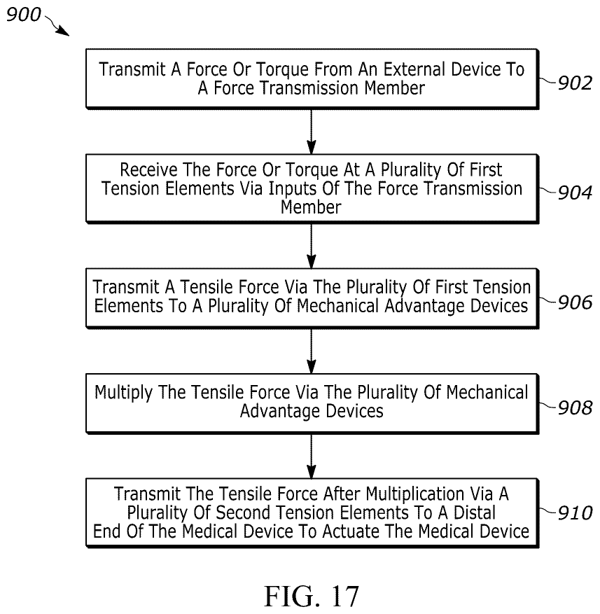

FIG. 17 illustrates a method 900 of articulating a medical device 600. At box 902, the method 900 includes transmitting a force or torque from an external device to a force transmission member 604. At box 904, the method 900 includes receiving the force or torque at a plurality of first tension elements 632 via inputs of the force transmission member 604. At box 906, the method 900 includes transmitting a tensile force via the plurality of first tension elements 632 to a plurality of mechanical advantage devices 630. At box 908, the method 900 includes multiplying the tensile force via the plurality of mechanical advantage devices 630. At box 910, the method 900 includes transmitting the tensile force after multiplication via a plurality of second tension elements 636 to a distal end 638 of the medical device 600 to actuate the medical device 600. The method 900 may further include any of the functionalities and features described above.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the foregoing description, one or more elements shown and described in association with one example, implementation, or application may be incorporated into other examples, implementations, or application unless specifically described otherwise, unless the one or more elements would make an example or implementation non-functional, or unless two or more of the elements provide conflicting functions. Similarly, it should be understood that any particular element, including a system component or a method process, is optional and is not considered to be an essential feature of the present disclosure unless expressly stated otherwise.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure.

While some examples are provided herein in the context of medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques may also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 112) or may be implemented, at least in part, in the form of executable code stored on non-transitory, tangible, machine-readable media that when run by one or more processors (e.g., processors of control system 112) may cause the one or more processors to perform one or more of the processes.

One or more elements in examples of this disclosure may be implemented in software to execute on a processor of a computer system such as control processing system. When implemented in software, the elements of the examples of the present disclosure are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one example, the control system supports wireless communication protocols such as Bluetooth, IrDA, HomeRF, IEEE 802.11, DECT, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the present disclosure are not described with reference to any particular programming language. It will be appreciated that a variety of programming languages may be used to implement the teachings of the present disclosure.

This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). The “pitch” direction and “yaw” direction are not necessarily limited to vertical and horizontal movement, respectively, but rather may be arbitrary directions orthogonal to one another. As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along a length of an object. As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a medical device that is closest to the target tissue would be the distal end of the medical device, and the end opposite the distal end would be the proximal end of the medical device.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial positions and orientations. The combination of a body's position and orientation defines the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, medical instrument, and variants thereof, can be interchangeably used.

While certain exemplary examples of the present disclosure have been described and shown in the accompanying drawings, it is to be understood that such examples are merely illustrative of and not restrictive on the broad disclosure herein, and that the examples of the present disclosure should not be limited to the specific constructions and arrangements shown and described, since various other modifications may occur to those ordinarily skilled in the art.

1. A medical device comprising:
a force transmission member including one or more inputs, the force transmission member configured to receive forces or torques from an external device;
an elongate member coupled to the force transmission member and extending distally from the force transmission member, the elongate member housing a plurality of first tension elements respectively coupled to the one or more inputs of the force transmission member and configured to receive and transmit a tensile force, the plurality of first tension elements comprising a first material;
a plurality of mechanical advantage devices configured to receive and multiply the tensile force, each mechanical advantage device coupled to one of the plurality of first tension elements and rotatable around a respective axis of rotation; and
a plurality of second tension elements configured to transmit the tensile force after multiplication of the tensile force to a distal end of the medical device, each second tension element coupled to one of the plurality of mechanical advantage devices and operatively coupled to the distal end of the medical device, the plurality of second tension elements comprising a second material that is different than the first material.

2. The medical device of claim 1, wherein the elongate member is flexible.

3. (canceled)

4. The medical device of claim 1, wherein the first material comprises one of a polymer and a metal, and the second material comprises the other of the polymer and the metal.

5. (canceled)

6. The medical device of claim 1, wherein each first tension element is coupled to one of the plurality of mechanical advantage devices a distance from the respective axis of rotation.

7. The medical device of claim 1, wherein at least one of the plurality of mechanical advantage devices is arranged in a series with a secondary mechanical advantage device and coupled to the secondary mechanical advantage device by an intermediate drive element to transmit and multiply the tensile force.

8. (canceled)

9. The medical device of claim 1, wherein the distal end of the medical device can be actuated manually when the medical device is decoupled from the external device.

10. The medical device of claim 1, further comprising a guide housing provided around the plurality of mechanical advantage devices and coupled to the elongate member and to the distal end of the medical device.

11. The medical device of claim 10, wherein the guide housing comprises a plurality of guide channels to direct movement of the plurality of mechanical advantage devices to translate within respective guide channels of the plurality of guide channels and parallel to a longitudinal axis of the medical device.

12.-14. (canceled)

15. The medical device of claim 10, wherein the guide housing comprises a central open passage.

16. The medical device of claim 1, wherein the medical device is a surgical instrument; and the distal end of the medical device is an end effector actuated by the plurality of second tension elements.

17. (canceled)

18. The medical device of claim 1, wherein the medical device is an endoscope; and the distal end of the medical device is an articulated bending section of the endoscope.

19.-20. (canceled)

21. The medical device of claim 1, wherein the plurality of mechanical advantage devices are arranged in antagonistic pairs.

22. The medical device of claim 21, wherein the medical device includes four antagonistic pairs to provide bidirectional motion along four axes.

23. A medical device comprising:
a force transmission member including one or more inputs, the force transmission member configured to receive forces or torques from an external device;
an elongate member coupled to the force transmission member and extending distally from the force transmission member, the elongate member housing a plurality of first tension elements respectively coupled to the one or more inputs of the force transmission member and configured to receive and transmit a tensile force;
a plurality of pulleys configured to receive and multiply the tensile force, each pulley rotatable around a respective axis of rotation and coupled to one of the plurality of first tension elements; and
a plurality of second tension elements configured to transmit the tensile force after multiplication of the tensile force to a distal end of the medical device, each second tension element coupled to one of the plurality of pulleys.

24. The medical device of claim 23, wherein each second tension element is coupled to the one of the plurality of pulleys at the respective axis of rotation by a tube having a clevis interface, the tube operatively coupled to the distal end of the medical device

25. The medical device of claim 23, wherein the plurality of first tension elements each has a first end operatively coupled to one of the one or more inputs of the force transmission member, a length wrapped around the respective pulley, and a stationary second end.

26. The medical device of claim 23, wherein the plurality of pulleys are configured to move axially, translating parallel to a longitudinal axis of the medical device.

27. (canceled)

28. The medical device of claim 23, wherein a mechanical advantage ratio of the tensile force provided by the plurality of second tension elements relative to the plurality of first tension elements is 2:1.

29. A medical device comprising:
a force transmission member including one or more inputs, the force transmission member configured to receive forces or torques from an external device;
an elongate member coupled to the force transmission member and extending distally from the force transmission member, the elongate member housing a plurality of first tension elements respectively coupled to the one or more inputs of the force transmission member and configured to receive and transmit a tensile force;
a plurality of levers configured to receive and multiply the tensile force, each lever rotatable around a respective axis of rotation and being coupled to one of the plurality of first tension elements at a first distance from the respective axis of rotation; and
a plurality of second tension elements configured to transmit the tensile force after multiplication of the tensile force to a distal end of the medical device, each second tension element being coupled to one of the plurality of levers at a second distance from the respective axis of rotation that is less than the first distance and a second end operatively coupled to the distal end of the medical device.

30. A medical device comprising:
a force transmission member including one or more inputs, the force transmission member configured to receive forces or torques from an external device;
an elongate member coupled to the force transmission member and extending distally from the force transmission member;
an end effector operatively coupled to the force transmission member;
a mechanical advantage device positioned between the end effector and the force transmission member; and
a tension element coupled to the end effector to drive articulation of the end effector, the tension element having a first end that is distal to the mechanical advantage device, a second end that is distal to the mechanical advantage device, and a proximal portion that is coupled to the mechanical advantage device.
